(19) United States
(12) Patent Application Publication
(10) Pub. No.: US 2001/0033664 A1
(43) Pub. Date: Oct. 25, 2001

(54) Hearing aid format selector

(75) Inventors: Christopher J. Poux, Trenton, NJ (US); Wayne J. Staab, Phoenix, AZ (US); Geary A. McCandless, Dammeron Valley, UT (US); Walter P. Sjursen, Washington Crossing, PA (US); Frederick J. Fritz, Skillman, NJ (US); Thomas J. Squeglia, Belle Mead, NJ (US); Gerald R. Iannelli, Marlton, NJ (US); Richard M. Moroney III, Princeton, NJ (US); Jennifer L. Semanchik, Lawrenceville, NJ (US); Sterling E. McBride, Princeton, NJ (US); Randall R. Tucker, West Windsor, NJ (US); Reuben Q. Zielinski, Belle Mead, NJ (US); John G. Aceti, West Windsor, NJ (US); Frederick Kelmer JR., Monroe Township, NJ (US)

Correspondence Address: Leo R. Reynolds, Esq., Hamilton, Brook, Smith & Reynolds, P.C., Two Militia Drive, Lexington, MA, 02421-4799, US

(73) Assignee: Songbird Hearing, Inc.

(21) Appl. No.: 09/808,099

(22) Filed: Mar. 13, 2001

Related U.S. Application Data
(60) Provisional application No. 60/188,996, filed on Mar. 13, 2000.
(60) Provisional application No. 60/208,634, filed on Jun. 1, 2000.

(51) Int. Cl. H04R 29/00; A61B 5/00

(52) U.S. Cl. 381/60; 600/559; 381/312

(57) ABSTRACT

In an illustrative embodiment of the present invention, a hearing aid device is tethered to a selector module via a cable. A keypad of the selector is pressed to program the hearing aid device with a particular acoustical format. More specifically, a command generated by the selector module is received at the hearing aid test unit to program a corresponding acoustical format. Consequently, a hearing impaired patient can reprogram the hearing aid device to compare which of multiple formats provides an optimal response for use.

### RELATED APPLICATIONS

This application claims the benefit of U.S. Provisional Application No. 60/188,996 filed on Mar. 13, 2000, the entire teachings of which are incorporated herein by this reference.

This application is related to copending U.S. Applications:

| ATTORNEY DOCKET NO. | APPLICATION NO. | TITLE |
|---|---|---|
| SMI-13459pA | 09/524,666 | Disposable |
| | | Modular Hearing |
| | | Aid |
| 2506.1005-001 | 09/524,043 | Mass Produced |
| | | Hearing Aid With |
| | | a Limited Set of |
| | | Acoustical |
| | | Formats |
| 2506.1013-001 | 09/524,040 | One-Size-Fits- |
| | | All Uni-Sar |
| | | Hearing |
| | | Instrument |
| 2506.2012-000 | 60/188,997 | Hearing Aid With |
| | | Flexible Shell |
| 2506.2013-000 | 60/188,996 | Hearing Aid |
| | | Prescription |
| | | Selector |
| 2506.2014-000 | 60/188,721 | Through-Hole |
| | | and Surface |
| | | Mount |
| | | Technologies for |
| | | Highly- |
| | | Automatable |
| | | Hearing Aid |
| | | Receivers |
| 2506.2019-000 | 60/188,857 | Remote |
| | | Programming and |
| | | Control Means |
| | | for a Hearing |
| | | Aid |

all filed Mar. 13, 2000, the entire teachings of which are incorporated herein by reference.

This application is also related to copending U.S. Applications:

| ATTORNEY DOCKET NO. | TITLE |
|---|---|
| 2506.2008-005 | Hearing Aid with Tinted |
| | Components |
| 2506.2019-001 | Remote Programming and |
| | Control Means for a Hearing |
| | Aid |
| 2506.1009-005 | Disposable Modular Hearing |
| | Aid |

all filed on even date herewith, the entire teachings of which are incorporated by reference.

### BACKGROUND OF THE INVENTION

The process of fitting hearing aids is not always an exact science. It is therefore desirable to easily and efficiently demonstrate characteristics of a hearing aid device such as sound quality for prospective users before dispensing a hearing aid for use.

Various master hearing aids and hearing aid programming devices have been used to dispense hearing aids. Until recently, most of the test units have been large table-top designs including panel-mounted microphones and headphones that have no relation to the transducers or circuitry actually used in the hearing aids to be fitted. Thus, a corresponding dispensed product to correct a hearing impaired patient typically falls short of a user's expectations and the patient has to revisit an audiologist for another fitting.

More recently, hand-held programming devices have been developed for fitting programmable analog and digital hearing aids. These devices typically enable an audiologist to adjust the response of a hearing aid device by changing one parameter at a time. This can be a complex procedure because it requires the audiologist to know the effect of changing each parameter.

### SUMMARY OF THE INVENTION

One aspect of the present invention is generally directed towards an apparatus and method for more efficiently manufacturing and dispensing hearing aid devices. In an illustrative embodiment, a hearing aid test unit is coupled to a selector module so that a user can select one of several acoustical formats to correct a hearing impairment of a user. Thus, a hearing impaired patient can select and compare a number of available acoustical formats of a hearing aid test unit to determine which setting is optimal for use.

One technique for coupling a hearing aid test unit to the selector module is to provide a cable for transmitting electronic signals form selector module to the hearing aid test unit to set an acoustical format. A connector such as a telephone connector is optionally disposed at one end of the cable to plug into the selector module while an opposite end of the cable can terminate with the hearing aid test unit.

Alternatively, a hearing aid test unit is coupled to the selector module via a wireless link. Thus, modulated data information transmitted by the selector module can be demodulated at a receiver unit disposed in the hearing aid test unit to set a particular format.

In one application, the hearing aid test unit is similar to a form and fit of a mass produced hearing aid device, which is programmed with a fixed acoustical format. A matrix of hearing aids, each of which is programmed with a fixed format, can be produced so that a group of hearing aid devices of a similar type provides a nearly identical acoustical response over a range of audible frequencies. Preferably, each of the mass produced hearing aid devices is programmed with one of multiple fixed acoustical formats. However, a dispensed hearing aid can be programmable also.

According to the principles of the present invention, a user can select a desired format using a programmable hearing aid test unit and selector module and later purchase a mass produced hearing aid device having a fixed format to correct a hearing impairment. An overall cost savings is realized since only a selected number of formats are supported and a dispensed hearing aid device and hearing aid test unit can otherwise include common components such as a microphone, speaker and shell.

An acoustical format optionally defines an acoustical response of a hearing aid device over an entire audible range of frequencies detectable by a human ear.

Although the hearing aid test unit can include a single test device for sampling an acoustical format for a hearing aid test unit disposed in one ear, two hearing aid test units can be provided so that both a right and left ear can be tested simultaneously. Thus, a first acoustical format can be selected to correct hearing in one ear while a second acoustical format can be selected to correct hearing in the other ear.

In one application, the hearing aid test unit is fitted into an ear and includes a disposable shell and tip. Thus, the shell and tip can be thrown away after use by a patient and a following patient can generally reuse the same hearing aid test unit but with a new shell and tip. This aspect of the invention ensures that germs are not transferred from one patient to another using the same hearing aid test unit. Preferably, the disposable tip of the hearing aid test unit is provided to direct amplified sound from a speaker into an ear canal of a user.

In another application, a core of the hearing aid test unit includes a speaker and microphone that is covered by a replaceable shell. A combination of the shell and core of the hearing aid test unit is preferably sized to fit in an ear of a hearing impaired patient while the selector module located within reach of user is used to select one of multiple acoustical formats for correcting a hearing impairment.

A tab is optionally provided for securing the shell to the core of the hearing aid test unit. After use by a patient, the shell can be removed by breaking the tab and separating the core from the shell. Consequently, a used shell will not ordinarily be reused since the broken tab indicates to the operator that the shell was previously used. Thus, another aspect of the present invention involves providing an indicator on a shell or other removably attached component of the hearing aid device to notify an operator that the component was previously used.

Selection of an acoustical format can be based on an input by a user such as a hearing impaired patient. Preferably, the input is a keypad that the user presses to select an acoustical format. A keypad can be a simple mechanism such as an electrical on/off switch that, when pressed, generates a signal for selecting an acoustical format.

The effectiveness of a particular format can depend on a type of sound to be amplified for a hearing impaired user. Multiple recordings are therefore optionally provided so that a user can determine which of multiple electroacoustical formats provides optimum hearing correction for a particular type of sound. For example, one type of sound can be classical music while another can be conversational speech.

As previously discussed, a selector module is provided to enable a user to select a particular acoustical format for the hearing aid test unit. One method of selecting a format is achieved by downloading digital information to program a corresponding acoustical format at the hearing aid test unit disposed in the ear of a user. For example, a digital signal indicating a particular format type can be downloaded to a target hearing aid test unit for selecting one of multiple formats supported by the hearing aid test unit.

Alternatively, a digital signal from the selector module to the hearing aid test unit can include multiplier coefficients or other digital information that is downloaded from the selector module to program a corresponding acoustical format. Thus, any type of suitable digital information can be downloaded to a programmable logic device of the hearing aid test unit to program an acoustical format. When used, a downloaded set of coefficient multipliers defines a frequency response of a digital filter of the hearing aid device.

Another embodiment of the present invention is generally directed towards a hearing aid test unit for fitting a patient with a production hearing aid. A hearing aid test unit preferably simulates a form and fit of a corresponding production hearing aid and includes an input such as a cable that provides an electronic signal for selecting a frequency response of the hearing aid test unit. Thus, a production hearing aid device normally programmed with a fixed frequency response can be reformatted based on electronic signals provided via the cable.

In one application, the hearing aid test unit is powered through the cable. Thus, space in the hearing aid test unit otherwise housing a battery can be alternatively used to house a cable termination.

Many of the features previously discussed can be individually and multiply combined with the hearing aid test unit to provide additional advantages. For example, a core of the hearing aid test unit can be housed by a shell including a detachable tip so that the core can be reused for testing other patients.

In a specific application, a matrix of acoustical formats is supported for selection by a user. For example, the matrix can include N×M formats where both N and M equal more than 2. Thus, one of four or more different frequency responses can be selected and tested by a hearing impaired user to correct a hearing impairment.

As shown in FIG. 1, both keypads 10, 15 include a 3×3 matrix of acoustical formats that can be selected. An extra key, namely the ENH key, is provided so that a patient can test an acoustical format in which acoustical input signals are amplified evenly across an audible frequency range. More specifically, both high and low audible frequency inputs are amplified to produce an overall volume enhanced acoustical output for a patient.

In a preferred application, an acoustical format defines a frequency response of a hearing aid device over the entire audible range of a human ear.

Another aspect of the present invention is generally directed towards an apparatus and method for removing or engaging a component of a hearing aid device. In an illustrative embodiment, the hearing aid device comprises a core including a microphone and speaker to sense and amplify a sound input for a hearing impaired patient. The hearing aid device preferably includes a removably attached component disposed in relation to the core of the hearing aid device, whereby the component includes a pull cord. Accordingly, the pull cord can be used to apply a force on the component in a particular direction.

In one application the removably attached component of the hearing aid device is a sheath that is pulled over the core of the hearing aid device to protect it from exposure to human tissue. The sheath can be replaced by pulling the cord to separate the sheath from the core. Consequently, a core hearing aid device can be reused by another patient without exposure to a previously used component such as the sheath. In a similar manner, a pull cord can be attached to a shell that houses the core of the hearing aid device.

In another application, the removably attached component of the hearing aid device is a disposable tip for directing sound into an ear of a hearing impaired patient. If the removably attached component such as a disposable tip accidentally breaks away from the hearing aid device during removal from an ear, the pull cord is used to remove the piece of the hearing aid lodged in an ear. That is, the component of the hearing aid device can be removed by pulling on the cord attached to the component. The pull cord can be dental floss or other suitable material.

As previously discussed, a pull cord can alternatively be provided so that a component can be secured to the core of the hearing aid.

In a more specific application, an indicator is provided on the removably attached device so that it is used only once. For example, a component such as a shell for housing the core of the hearing aid device can include a locking mechanism to secure the shell to the core. When the shell is removed for a following patient the hearing aid test unit, a portion of the locking mechanism is designed to break away from the shell to provide an indication that the shell was previously used.

Another aspect of the present invention is generally directed towards an apparatus and method for more efficiently providing an electrical connection between two conductive nodes in a circuit. In an illustrative embodiment, an electrically conductive lead comprises a conductive strip that includes a slit for attaching the lead to an electrode such as a protruding terminal. Thus, the lead can be removably attached to the terminal.

One method of providing a slit on the strip is to etch away material on the strip. Alternatively, the slit can be stamped into the strip with a machine press or the like.

Although a shape of the slit can vary, preferably the slit is shaped to include at least one tongue. Consequently, the lead will remain securely fastened to a terminal based on resistive forces of the tongue on the terminal. The slit can also be shaped like a letter H, forming two tongues.

The strip is preferably a flat, length of metal that is rolled at one end to form a curl. The curl can provide a spring-like action for providing contact to a second electrode. Of course, tension of the spring will depend on the type and thickness of conductive strip. Other suitable shapes other than a curl can be formed at the end of the strip for connection to an electrode.

One specific use of the electrically conductive lead is to provide connectivity between a terminal of an electronic component to other electronic circuitry disposed within a hearing aid device.

Another aspect of the present invention is generally directed towards an apparatus and method for more efficiently coupling to an electronic circuit of a hearing aid device for processing an acoustical input. A connector is attached to the electronic circuit to form an assembly, around which a shell is formed to produce a hearing aid device that fits in an ear. Consequently, it is a simple procedure to provide connectivity to the electronic circuitry through, for example, a cable attached to the connector.

In one application, the connector is a surface mount connector to reduce size. A connector attached to the electronic circuit can include a post to which at least one wire is soldered. Alternatively, the connector includes pin receptacles so that it can be joined with a matable connector such as a socket.

The electronic circuit attached to a connector optionally includes an amplifier to amplify an acoustical input of the hearing aid. Thus, an acoustical input signal can be processed by the electronic circuit and then transmitted through the connector to a target device. To provide stability, the electronic component itself can be attached to a transducer of the hearing aid. Generally, an assembly of transducer, electronic circuit and connector is easier to maneuver for assembly in a corresponding hearing aid device.

In one application, the transducer is a microphone to which the electronic circuit is attached and a speaker is included in the hearing aid device for producing an acoustical output.

Another aspect of the present invention is generally directed to an apparatus and method for providing a more robust hearing aid test unit for testing a hearing impaired patient. Generally, a cable assembly such as a cable and connector is terminated at one end by a hearing aid test unit and electronics for processing an acoustical input signal of the hearing aid test unit are processed by an electronic circuit disposed in the cable assembly to produce an acoustical output. Consequently, the hearing aid device can be smaller since the electronic circuitry for processing an acoustical signal can reside in a cable attached to the hearing aid test unit.

The electronic circuit can be disposed almost any where in the cable assembly. For example, the electronic circuit can be disposed in a housing at a point along a length of the cable assembly or even in the cable itself. Alternatively, the electronic circuit can be disposed in a connector at the end of the cable assembly opposite the hearing aid device. In space restricted applications, the electronic circuit can include a flexible circuit board disposed in the cable assembly.

The cable assembly including hearing aid device can be connected to a selector module for selecting a mode of the electronic circuit and acoustical response of the hearing aid device. Thus, a user can select an acoustical response of the hearing aid device by providing an input to the selector module. More specifically, a user can press a key of the selector module to select an acoustical response of the hearing aid device. Preferably, the cable assembly includes a connector at an end opposite the hearing aid test unit for connection with the selector module.

In a specific application, a microphone is disposed in the hearing aid device for detecting an acoustical input and a corresponding signal is transmitted over at least a portion of the cable assembly to the electronic circuit for processing. Thus, circuitry otherwise disposed in the hearing aid device can be disposed in the cable assembly. A speaker is optionally included at a hearing aid device that is driven by the electronic circuit to produce an amplified output signal.

As previously discussed, the electronic circuit can be programmed to support a particular selected acoustical format. Although a format can define an acoustical response for a specified range of frequencies, an acoustical response or format of the hearing aid device can define an acoustical response for an entire range of audible inputs detectable by a human ear. Consequently, it is a simple process to fit a user with a proper hearing aid programmed with an appropriate acoustical format because there are fewer settings to choose a format.

Generally electronic circuit can include different types of circuitry to process an acoustical input signal. For example, the electronic circuit can include an amplifier and filter circuit for processing an acoustical input of the hearing aid device to generate an acoustical output of the hearing aid device. Consequently, a transducer such as a microphone disposed in the hearing aid can generate a signal that is processed at the electronic circuit disposed in the cable assembly.

To provide strain relief, shield wires of the cable assembly can be terminated in the hearing aid device by being attached to a component such as a shell of the hearing aid device. Depending on the application, the shield can be soldered, glued or welded to a shell of the hearing aid device.

An acoustical response of the hearing aid disposed at an end of the cable assembly can be trimmed so that an acoustical output of the hearing aid device conforms to a standard. Thus, the electronic circuit of the hearing aid device can be tested and programmed to compensate for a variation of a component disposed in the hearing aid device. For example, a microphone and speaker component of the hearing aid device can be tested to determine corresponding trim information that to be stored in memory of the electronic circuit to compensate for an identified component variation.

As previously discussed a tethered hearing aid device including the cable assembly can be reprogrammed by a user to select an acoustical format. To correct for a hearing impairment based on a selected acoustical format, a corresponding untethered hearing aid device programmed to a fixed format is dispensed to the patient for use. Consequently, a dispensed hearing aid device programmed to a fixed format can be provided at a lower cost to a consumer because such a device can be mass produced. In a more specific application, the untethered hearing aid device is an untethered ear piece that fits comfortably in an ear and is disposable to reduce the hassles associated with repeatedly cleaning the device.

Another aspect of the present invention is generally directed toward an apparatus and method for more efficiently assembling small components disposed in a hearing aid device that fits in an ear. Generally, a portion of an electronic component is formed to engage with a non-conductive portion of a corresponding socket. When combined, it is a simpler process to assemble a hearing aid device to include the socket and component. A component is optionally a transducer such as a microphone having a conductive body.

Typically, an electronic component includes contacts such as protruding conductive terminals. Thus, a socket can include receptacles for receiving the terminals.

Socket and component are potentially attached in two ways. First, a non-conductive body of the socket and component can be engaged. Second, conductive parts of the component and socket can be engaged to provide electrical connectivity between the component and other circuitry to which the socket is connected.

To ensure that the component and socket are not accidentally misaligned during assembly, conductive terminals can be asymmetrically disposed so that each conductive terminal is plugged into a corresponding receptacle when the socket and component are engaged. Consequently, an electronic component otherwise damaged during assembly will be protected.

In one application, the socket is cylindrically shaped and includes a terraced step for engaging with the component. Based on this combination, a component such as a microphone is more easily mounted in a hearing aid device.

Receptacles in the socket are optionally pins that extend through a non-conductive body of the socket so that a lead can be further attached to a particular pin.

According to the aspects of the present invention as previously discussed, the odds of successfully dispensing a hearing aid are greatly improved by providing a test unit with selectable acoustical formats that is a form, fit and function of a hearing aid device actually dispensed to a patient. The success rate of dispensing a proper hearing aid device is even further improved since the end user can participate in the format selection process.

One method of the present invention as previously discussed is a simpler method for dispensing hearing aids because a user can select only one of a limited set of pre-programmed acoustical formats to correct a hearing impairment. Thus, it is not necessary to adjust an excessive number of parameters of the hearing aid device to correct a hearing impairment of a user these processes are typically inaccurate due to the complexity associated with adjusting such parameters.

### BRIEF DESCRIPTION OF THE DRAWINGS

FIG. 1 is a diagram of a hearing aid test unit and selector module according to certain principles of the present invention.

FIG. 2 is a block diagram of a selector module for selecting an acoustical format according to certain principles of the present invention.

FIG. 3 is a detailed block diagram of a selector module according to certain principles of the present invention.

FIG. 4 is a detailed block diagram of a selector module according certain principles of the present invention.

FIG. 5 is a graph illustrating a set of acoustical formats according to certain principles of the present invention.

FIG. 6 is a block diagram of an electronic circuit that supports multiple selectable acoustical formats according to certain principles of the present invention.

FIG. 7 is a cross-sectional view of a hearing aid test unit according to certain principles of the present invention.

FIG. 8 is a cross-sectional view of a core hearing aid test unit according to certain principles of the present invention.

FIG. 9 is a cross-sectional view of a disposable shell and tip for housing a core of a hearing aid test unit according to certain principles of the present invention.

FIG. 10 is a cross-sectional view of a removably attached mushroom tip according to certain aspects of the present invention.

FIG. 11 is a cross-sectional diagram of a removably attached component according to certain principles of the present invention.

FIG. 12 is a diagram of an electrically conductive lead according to certain principles of the present invention.

FIG. 13 is a diagram of a conductive lead attached to a terminal of a component according to certain principles of the present invention.

FIG. 14 is a cross-sectional view illustrating how an electrically conductive lead is utilized in a hearing aid device according to certain principles of the present invention.

FIG. 15 is a cross-sectional diagram of a connector attached to an electronic circuit board component according to certain principles of the present invention.

FIG. 16 is a block diagram of an electronic circuit disposed in a cable assembly for processing an acoustical input signal according to certain principles of the present invention.

FIG. 17 is a more detailed block diagram of a cable assembly including a hearing aid test unit disposed at one end according to certain principles of the present invention.

FIG. 18 is a block diagram of a tester unit for trimming a cable assembly and hearing aid test unit according to certain principles of the present invention.

FIG. 19 is a 3-D view of a cylindrically shaped socket to which a hearing aid component is mounted according to certain principles of the present invention.

FIG. 20 is a cross-sectional diagram of a microphone for attachment to a socket according to certain principles of the present invention.

FIG. 21 is a cross-sectional diagram of a shielded cable termination in a hearing aid device according to certain principles of the present invention.

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

### DETAILED DESCRIPTION OF THE INVENTION

A description of preferred embodiments of the invention follows.

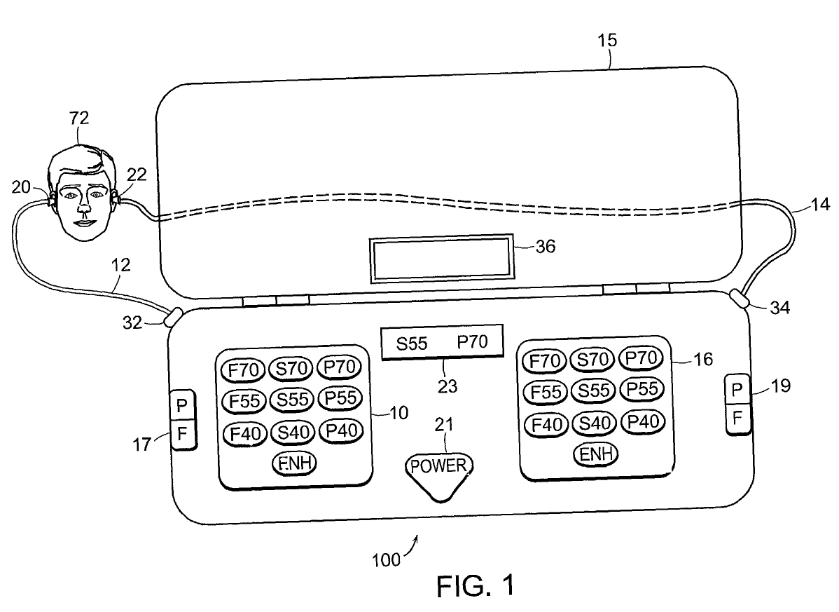

FIG. 1 is a diagram of a hearing aid prescription selector according to certain principles of the present invention. Selector module 100 includes electronic circuitry for enabling a user to select one of multiple electroacoustic formats of hearing aid test units 20, 22 disposed in ears of hearing impaired patient 72. Consequently, patient 72 can compare acoustical formats and determine an optimal format for use.

As shown, selector module 100 includes a left set of keypads 10 and a right set of keypads 16. Nine of the keypads include an alphanumeric label, each corresponding to a unique electroacoustical format for potentially correcting a hearing impairment of patient 72. To program an acoustical format of right hearing aid test unit 22, a user presses a key on right keypad 16. For example, key F70 is pressed to select an acoustical format corresponding with F70 in right hearing aid test unit 22. When a key is pressed, an electronic signal generated by electronics within selector module 100 is transmitted through cable 14 to right hearing aid test unit 22.

The information transmitted to hearing aid test units 20, 22 for selecting an acoustical format can vary depending on an application. For instance, the transmitted information is preferably digitally encoded data identifying which of multiple acoustical formats should be programmed in corresponding hearing aid test units 20, 22.

In one application, the information transmitted on cable 14 is simply encoded data that is a command for selecting which of nine pre-programmed acoustical formats is to be programmed at hearing aid test units 20, 22. Generally, an acoustical format defines a frequency response of the hearing aid device across an audible range of acoustical inputs. In one application, an acoustical format defines an acoustical response of an entire range of frequencies detectable by a human ear.

Aspects of a potential set of acoustical formats is discussed in more detail in “Satisfying Patient's Need with Nine Fixed Acoustical Prescription Formats,” G. McCandless, et al., Hearing Journal, Volume 53, #5, May, 2000:42-50, the entire teachings of which are incorporated herein by reference.

Generally, the parameters supporting an acoustical format reside in circuitry of the hearing aid test units 20, 22. For example, more details of electronics in hearing aid test unit is described in co-pending U.S. Application No. (2506.2019-001), filed on an even date herewith, entitled “Remote Programming and Control Means for a Hearing Aid,” the entire teachings of which are incorporated by reference. Upon receipt of a selection command, a new mode is programmed at a target hearing aid test unit 20, 22 to provide the newly selected format. That is, the electronic circuitry of the target hearing aid test unit 20 is reprogrammed so that a sound input at a microphone of the unit is properly amplified to produce an appropriate sound output from a speaker into ear of patient 72 based on the selected acoustical format.

In an alternative embodiment, selector module 100 transmits a set of parameters for programming a target hearing aid test unit 20, 22. For example, a set of multiplier coefficients for a digital filter disposed within core electronics of hearing aid test unit 22 is transmitted over cable 14. Based on either method, patient 72 can individually program left or right hearing aid test unit 20, 22 by merely pressing a key of left keypad 10 or right keypad 16, respectively.

As shown, selector module 100 can include a display 23 such as an LCD display to indicate the acoustical format presently programmed for each hearing aid test unit 20, 22. Consequently, a patient 72 can compare each of multiple acoustical formats and identify which format is most desirable for use.

A hinged cover 15 is optionally provided for protecting keypads 10, 16 of selector module 100. PF (Previous Format) toggle buttons 17, 19 are disposed at the edges of selector module 100 so that they are accessible even when cover 15 is closed.

Generally, PF buttons 17, 19 are provided so that a patient 72 can more easily compare acoustical formats. For example, pressing left PF button 17 enables a patient 72 to quickly switch between two previously selected formats for comparison purposes. Based on the quality of sound provided by each format, a patient 72 can more easily determine an optimal format for use in a dispensed hearing aid programmed with a fixed format.

Hinged cover 15 includes a window 36 so that patient 72 can identify which of multiple formats is selected even when cover 15 is closed.

Power switch 21 is also protected by cover 15 when closed so that the selector module 100 is not accidentally turned off during use. Preferably, power switch 21 is an on/off switch for connecting power of a battery source to electronic circuitry also located in the selector module 100. Thus, selector module 100 can be portable so that a patient 72 can test different formats in real world settings.

Cable 12, 14 is optionally a wireless link so that a patient 72 is not tethered to selector module 100. Details of this embodiment are described in U.S. application No. (Docket 2506.2019-001), previously incorporated by reference. This wireless aspect of the present invention enables a user to move about freely without concern for damaging the hearing aid test units 20, 22 by pulling on cords 12, 14 respectively.

In an embodiment where a hearing aid test unit 20 is coupled to selector module 100 via a wired link, a connector 32 is optionally disposed at end an of cable 12. Consider that the life of a hearing aid test unit 20 is potentially limited because of stress caused by successive use by patients 72. That is, a wire can eventually break off the hearing aid test unit 20 as a result of continued use. When a hearing aid test unit is damaged, it can be thrown away and replaced by a new unit. To support reuse of selector module 100, cable 12 can include a connector 32 that plugs into selector module 100. In a specific application, the connector is a telephone type jack/connector that is generally available at low cost and easily produced. In an alternate application, connector 32 is a DIN connector.

As previously discussed, multiple acoustical formats can be sampled via hearing aid test units 20, 22. For example, a column of left keypad 10 includes an N×M matrix, where N represents the number of formats in a column and M represents a number of formats in a row. In the embodiment as shown, N=3 and M=3. Although any size matrix can be used, preferably both N and M are equal to more than 2.

Acoustical formats of hearing aid test units 20, 22 are preferably similar to those provided in a corresponding production hearing aid device available in a fixed format. In one application, a dispensed hearing aid programmed with a fixed format is disposable so that it is no longer necessary to repeatedly clean the device after extended use. A patient 72 can therefore identify a desirable format and purchase a corresponding production hearing aid device to correct a hearing impairment.

A hearing aid test unit 20, 22 having a general shape to fit a typical user can include programmable electronics and similar components such as a microphone, speaker, and shell that are used in a production hearing aid having a fixed format. Similarly shaped hearing aid devices can thus be programmed and trimmed to produce a family of hearing aids having nearly identical acoustical qualities. That is, a family of hearing aid devices such as F70 includes hearing aids that are programmed and trimmed in production to have a similar frequency response to acoustical formats supported by hearing aid test units 20, 22 as selected by a hearing impaired patient 72.

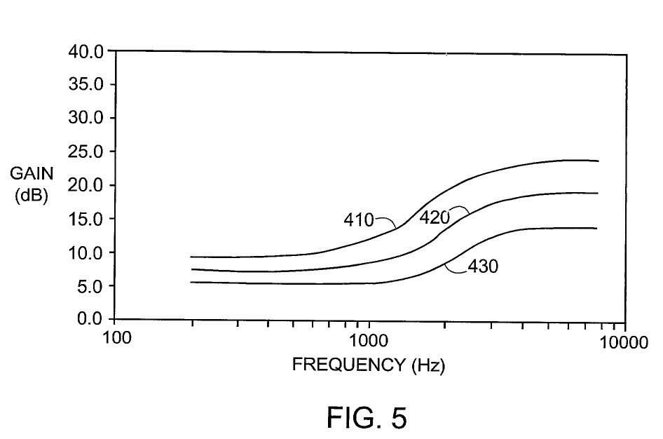

FIG. 5 more particularly illustrates a set of potential acoustical formats for correcting different types of hearing impairments. As shown, line 410 corresponds with acoustical format F70, line 420 with F55 and line 430 with F40. More details regarding the acoustical response of fixed format production of hearing aids is described in U.S. application Ser. No. 09/524,043, entitled “Mass Produced Hearing Aids with a Limited Set of Acoustical Formats,” filed on Mar. 13, 2001, the entire teachings of which are incorporated by reference. Based on the previously discussed method of selecting an appropriate acoustical format, patient 72 can select an acoustical format for either hearing aid test unit 20, 22 and later purchase a low-cost production hearing aid device having the same acoustical format.

One aspect of left keypad 10 and right keypad 16 is the classification of acoustical formats. For instance, a row of keys including F55, S55, and P55 can correspond with acoustical formats that vary in terms of treble and base amplification. More particularly, keys on a left side of a row can correspond with acoustical formats that provide more amplification at bass frequencies. Conversely, keys on the right hand side of a row can correspond with acoustical formats that provide less amplification at bass frequencies. Thus, a user can more precisely select an appropriate acoustical format to correct a hearing impairment without having to randomly press buttons to select an acoustical format.

In a similar manner, a column of keys such as F70, F55 and F40 can correspond with acoustical formats that provide more or less overall gain. For example, a format such as F70 located at a top of a column correspond with acoustical formats providing higher overall gain while the formats such as F40 located at a bottom of a column can correspond with acoustical formats providing lower overall gain across a range of audible frequencies. Consequently, patient 72 can select a format towards a top of a column to increase a loudness level of hearing aid test unit 20, 22. Conversely, a patient 72 can select a format towards a bottom of a column if a previous format is too loud. Based on this technique, patient 72 can more easily identify a preferred acoustical format for later use.

Overall operation of selector module 100 and use of hearing aid test units 20, 22 is simple enough for the prospective patient 72 to select and compare different acoustical formats in real world settings. Specifically, patient 72 can carry a portable selector module 100 around almost anywhere so that a user can test an acoustical format in settings where the hearing aid device will normally be used.

A battery is optionally included in each hearing aid test unit 20, 22 so that patient 72 can disconnect a corresponding cable 12, 14 after a format is programmed. In such an embodiment, patient 72 would be free to move about without being tethered to the selector module 100. Preferably, a power source such as a rechargeable battery resides in the hearing aid test unit 20, 22. To reprogram a format, a corresponding cable 12, 14 could then be re-connected to the hearing aid test unit 20, 22 and an appropriate keypad would be pressed by the user to select another format.

In another embodiment, cable 12, 14 is optionally fixed to the hearing aid test unit 20, 22 and power is provided to the hearing aid test unit via a power supply controlled at selector module 100.

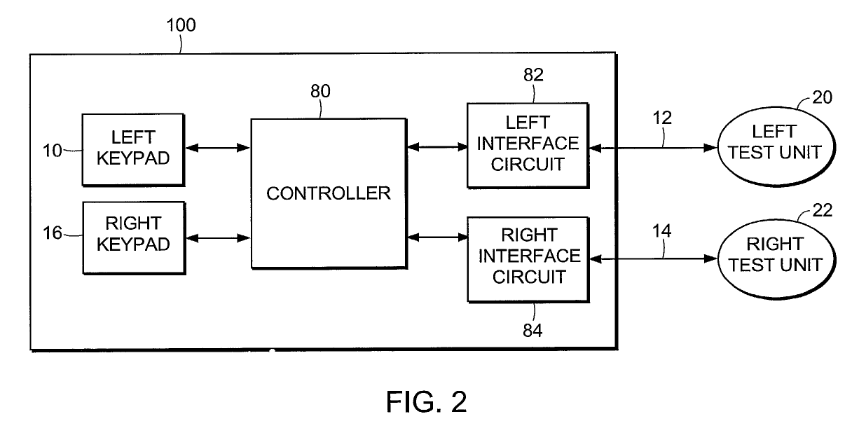

FIG. 2 is a block diagram selector module and a pair of tethered hearing aid test units. Left keypad 10 and right keypad 16 are coupled to controller 80, which senses, inter alia, when a key is pressed. An appropriate command is then generated and transmitted to the appropriate hearing aid test unit 20, 22 via left or right interface circuit 82, 84. Both interface circuits 82, 84 preferably support a digital protocol for transmitting data information to corresponding hearing aid test unit 20, 22.

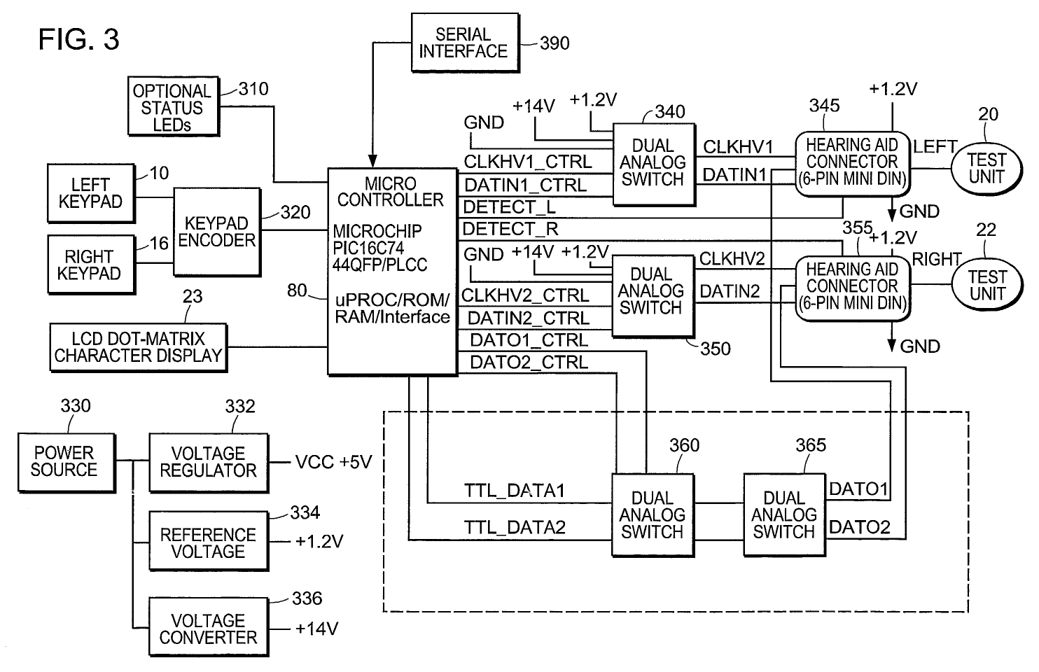

FIG. 3 is a detailed block diagram illustrating one embodiment of selector module 100 according to certain principles of the present invention.

Power source 330 such as a battery or wall power input provides a voltage that is used to essentially power selector module 100 and, optionally, hearing aid test units 20, 22 as mentioned. Voltage regulator 332 provides a Vcc voltage of +5 volts. Reference voltage generator 334 produces a 1.2 volt reference for A/D and D/A converters. Lastly, voltage converter 336 converts a voltage input from power source 330 to +14 volts for corresponding circuitry of selector module 100.

Controller 80 is preferably a microcontroller device including a microprocessor, read-only memory, volatile memory, and interface circuitry for communicating or driving circuitry such as optional status LEDs 310. Left keypad 10 and right keypad 16 are coupled to keypad encoder 320, an output of which is fed to controller 80 for sensing whether a key has been pressed by a user.

Upon detection of a pressed key, controller 80 generates data information that is transmitted to a target hearing aid test unit 20, 22 for programming a selected acoustical format. Preferably, data is transmitted to a target hearing aid test unit 20, 22 by driving a clock and data line emanating from dual analog switch 340, 350 through connector 345, 355. Additional electronic signals generated by hearing aid test unit 20, 22 are driven through cable 12, 14 to controller 80 to support communication in the reverse direction.

Generally, any protocol can be used for transmitting a program selection command to hearing aid test unit 20, 22. In the present application, a data line is driven with a data bit and a clock signal is toggled to program a target hearing aid test unit 20, 22.

Serial interface 390 enables outside connectivity with selector module 100 and, more particularly, controller 80. For example, serial interface 390 can be used to interface controller 80 with a PC (Personal Computer) or other digital device. Consequently, the code running on controller 80 can be downloaded from a personal computer or other programming device.

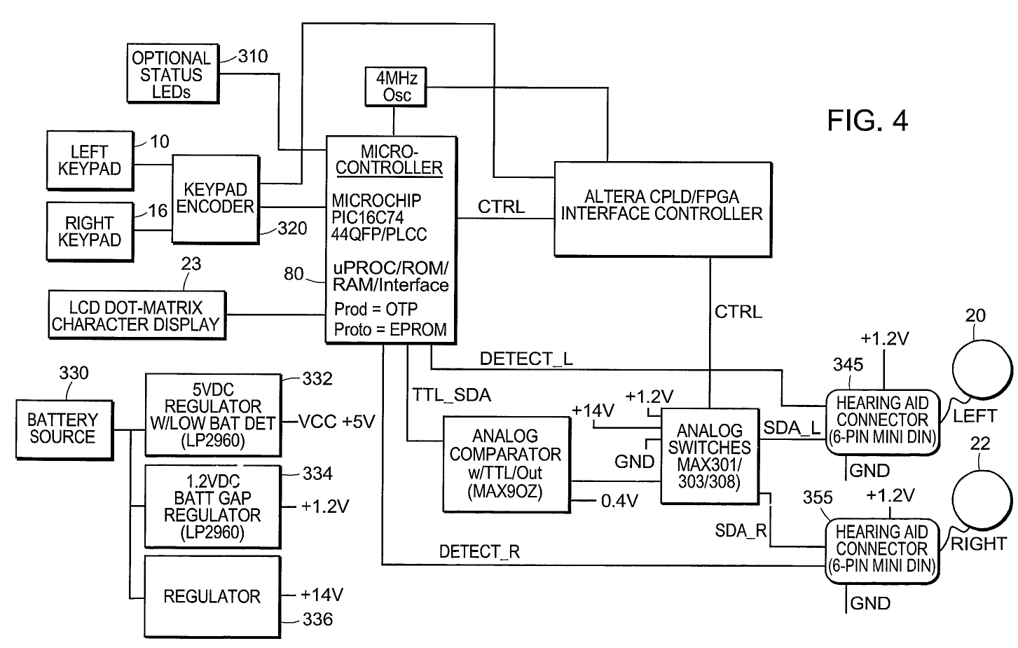

FIG. 4 is another detailed block diagram of electronics potentially located in selector module 100. The schematic is generally the same as previously disclosed. However, an FPGA (Field Programmable Gate Array) replaces several chips and is programmed with interface controller logic 470 for supporting communications with hearing aid test units 20, 22.

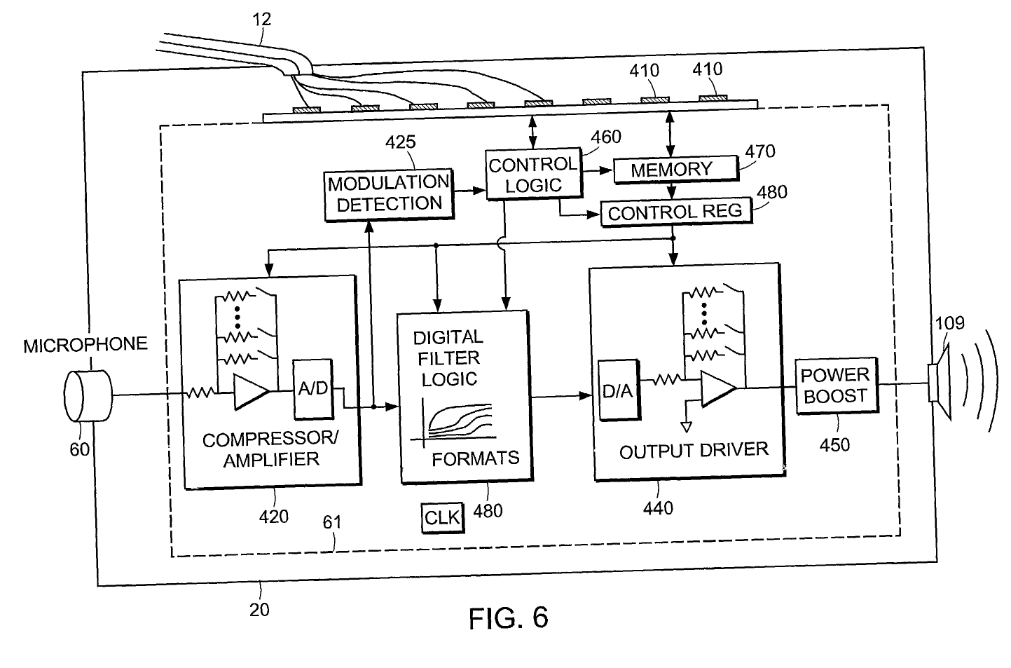

FIG. 6 is a block diagram more particularly illustrating a hearing aid test unit according to certain principles of the present invention.

Hearing aid test unit 20 includes a microphone 60 for detecting an acoustical input signal. An output of the microphone 60 is fed to electronic circuit 61 for processing. Generally, the signal is amplified at compressor/amplifier stage and is filtered via digital filter logic 430. A digital output of the filter logic 430 is then fed to output driver 440 that converts the digital input back to an analog signal for driving speaker 109.

One aspect of the present invention involves testing electronic circuitry 61 and related components so that hearing aid test units 20, 22 provide an acoustical response that conforms to a standard. That is, the sound output generated by one hearing aid test unit 20 is preferably the same for a particular acoustical as the sound generated by another hearing test unit 22.

As shown, microphone 60 is coupled to electronic circuit 61 including multiple stages of analog and digital circuitry. Test points are chosen at various stages of electronic circuit 61 so that it can be tested by a tester. For instance, an output of the amplifier/compressor stage 420 is connected to test pad 410 so that the corresponding signal can be measured at a component tester when the electronic circuit 61 is put in a test mode.

To test hearing aid test unit 20, 22, discussed, a controlled acoustical input is provided at microphone 60 and a generated signal is amplified by amplifier/compressor circuit. The amplified signal is then measured at a component tester to determine how its characteristics deviate from a standard. Based on the measured deviation, a compensation factor is programmed into memory 470 of electronic circuit 61. This is achieved by transmitting a compensation factor in the form of digitally encoded data from component tester to memory 470. Control logic 460 includes hardware to support the data transfer into memory device 470.

Multiple stages of the electronic circuit 61 can be analyzed so that multiple compensation factors are stored in memory device 470, each of which is used to trim an aspect of circuit 61. For example, one compensation factor is downloaded into memory 470 to compensate for an overall response of the hearing aid device.

To support testing, control logic 460 enables certain circuits to be bypassed. For example, digital filter 430 can be placed in a by-pass made so that only specific portions of a circuit are tested. Thus, it is possible to isolate a stage of circuit 61 and determine an appropriate compensation factor that should be programmed to compensate for a particular aspect or stage of circuit 61.

As previously discussed, hearing aid test units 20, 22 are disposed at an end of corresponding cables 12, 14. After trimming, cable 12 is attached to hearing aid test unit 20. Digital information is transmitted through the cable 12 to memory 470 for programming an acoustical format. This data is then latched into control register 480 that drives analog and digital stages of electronic circuit 61 to program a selected acoustical format.

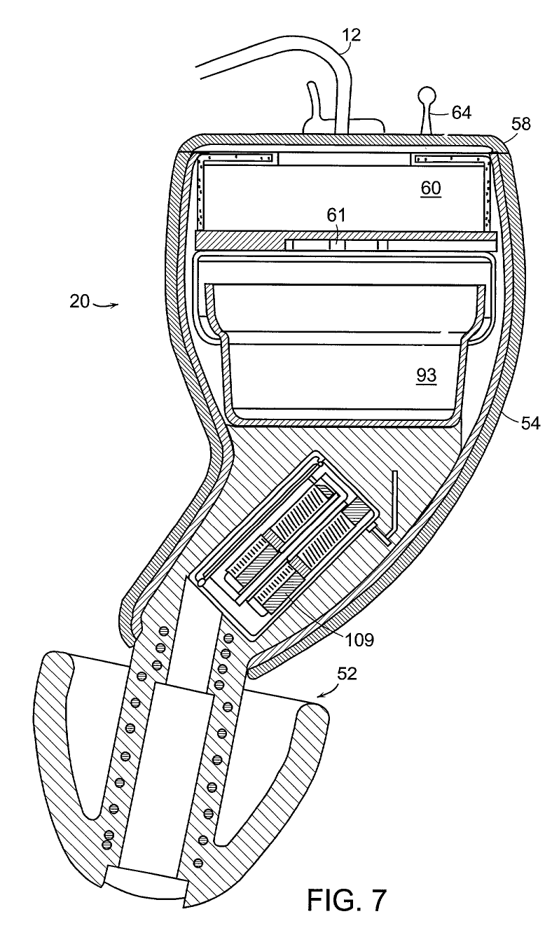

FIG. 7 is a cross-sectional view of a hearing aid test unit according to certain principles of the present invention. As shown, components disposed in the hearing aid test unit 20, 22 are housed by shell 54. Although shell 54 can be almost any material, it is preferably made of a plastic-type material.

Generally, hearing aid test unit 20 can resemble a corresponding production-type unit that is dispensed with a fixed format as previously discussed. Thus, both a production hearing aid device and can include the same type of components. For example, each device can include a similar speaker, microphone and electronics. However, a cable is provided between the selector module 100 and hearing aid test unit 20 so that an acoustical format can be reprogrammed.

Battery 93 provides power to electronics 61 that amplifies sound input received at microphone 60. The processed signal is then used to drive speaker output device 109. One difference between hearing aid test unit 20, 22 and dispensed hearing aid device is cable 12 attached to hearing aid test unit 20 at microphone cover or lid 58. This is provided so that hearing aid test unit 20 can be reprogrammed as previously discussed.

In an application where the hearing aid test unit 20 is powered through cable 12, battery 93 can be unpopulated.

Tip 52 of hearing aid test unit 20 is preferably made of a soft rubber-like material so that, when inserted, it molds to a shape of patients 72 ear canal. Preferably tip 52 is soft and shaped like a mushroom so that it fits snugly.

In one application, hearing aid test unit 20 is designed for re-use. For example, a core 50 of hearing aid test unit 20 is removable so that shell 54 and tip 52 can be replaced for each new patient 72. Cord 64 enables a user to pull core 50 out of shell 54 and, after testing, pull hearing aid test unit 20 out of patient's ear canal.

Shell 54 is similar to production clam shell described in U.S. patent Ser. No. 09/524,040, filed on Mar. 13, 2000, entitled “One-Size-Fits-All Uni-Ear Hearing Instrument,” the entire teachings of which are incorporated herein by reference. However, lid 58 is cutaway from so that shell 54 is replaceable. It should be noted that shell 54 is optionally multiple interconnected pieces or a single piece construction.

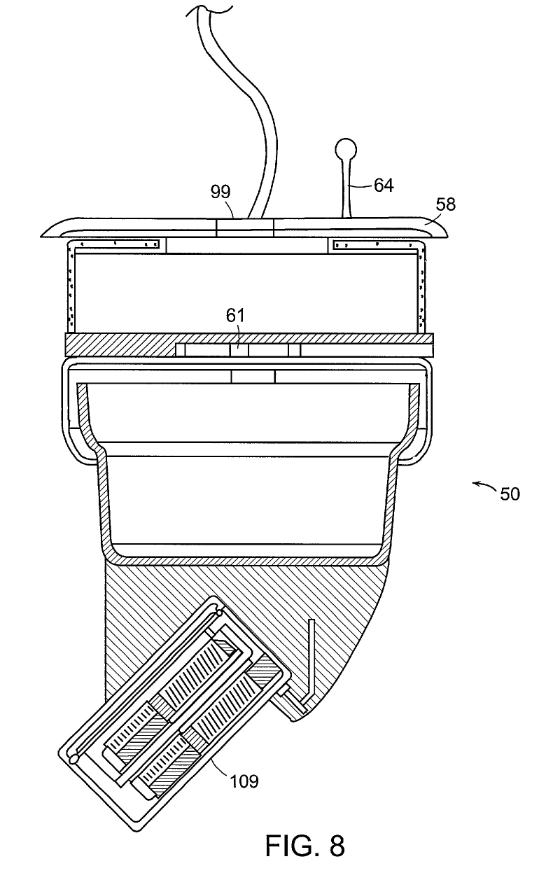

FIG. 8 is a cross-sectional view of a core hearing aid test unit according to certain principles of the present invention.

Core 50 optionally includes microphone 60, battery 93, electronics 61 including a digital filter and amplifier circuit, and speaker 109. Since core 50 is an assembly, its components can be held together via an adhesive such as epoxy or silicon rubber. Lid 58 is glued to microphone 60 so that the combination of core 50 and shell 54 appear similar to a mass-produced hearing aid rather than a test unit. Thus, hearing aid test unit 20 generally provides the same form, fit and function as a finally dispensed product.

As previously discussed, lid 58 includes a pull cord 64 made of wire braid, mesh, plastic or other suitable material so that core 50 can be extracted from an ear of patient 72 or disposable shell 54.

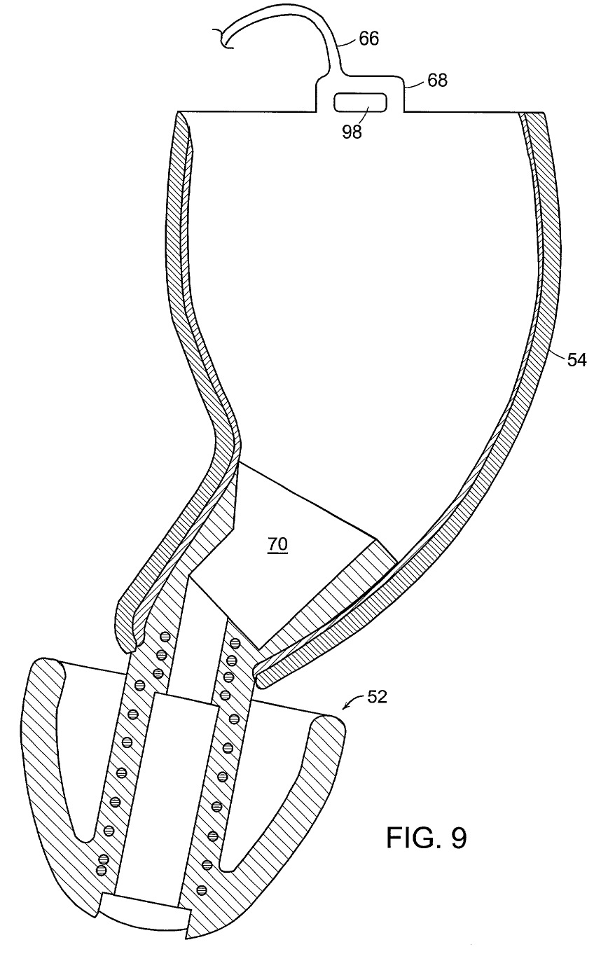

FIG. 9 is a detailed cross-sectional view of a shell and tip. A nest 70 for speaker 109 of core 50 is optionally shortened to allow easier coupling of core 50 into shell 54. Two shell halves forming shell 54 including tip 52 are optionally supplied to an audiologist dispenser as a complete assembly.

Shell 54 can include a lock mechanism 68 to secure core 50 and shell 54. More specifically, tabs 99 disposed on core 50 can fit through opening 98 of lock mechanism 68. Tabs extending axially from lid 58 fit through opening 98 of lock assembly 68 so that shell 54 is removably attached to core 50. After use, the lock mechanism 68, preferably made from plastic, can be broken to remove old shell 54 and tip 52 from hearing aid test unit 20.

Lock mechanism 68 thus serves multiple purposes. For instance, it is used to secure core 50 to shell 54. When broken, lock mechanism 68 provides an indication that the shell 54 was previously used. Thus, breaking the lock mechanism 68 prevents accidental reuse of shells 54. Additionally, if a user attempt to reuse a shell 54 with a broken lock mechanism 68, lock mechanism 68 will not necessarily secure shell 54 to core 50 since it is broken from previous use.

Based on this technique of re-using a core and replacing core 50 and tip 52, there is generally little health risk caused by using the same hearing aid test unit 20, 22 for multiple patients. Hearing aid test unit 20, 22 would otherwise have to be discarded or cleaned for re-use. Discarding the hearing aid test unit 20, 22 is undesirable because of the expense to replace it. Cleaning the hearing aid test unit is also undesirable because it is often difficult to control how thoroughly a test unit 20, 22 is cleaned and the process of cleaning is time-consuming since it is an electronic part.

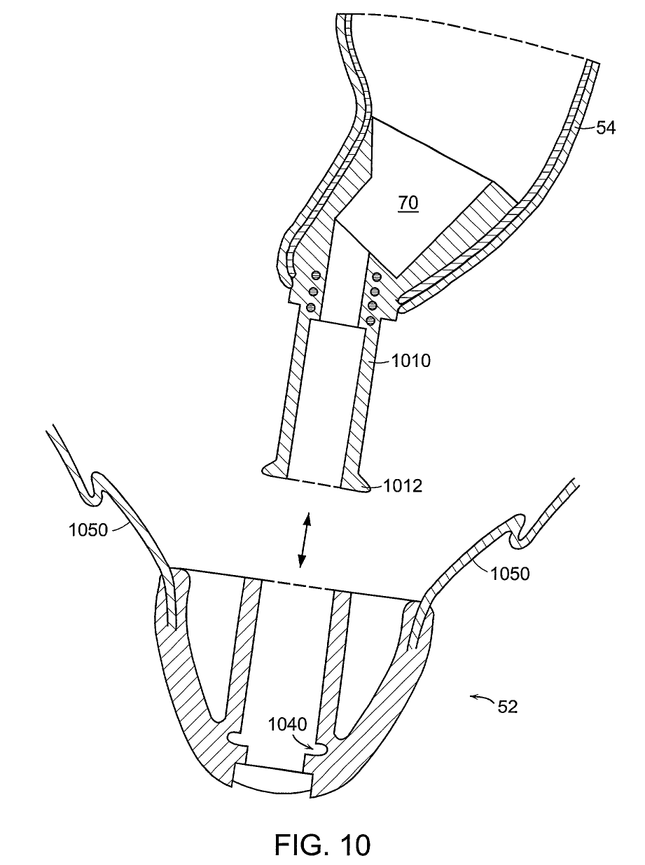

FIG. 10 is a cross-sectional view of a disposable shell and tip according to certain principles of the present invention. In the embodiment as shown, shell 54 and tip 52 are provided as removably attached components.

Nest 70 for receiving speaker 102 can include a neck 1010 and lip 1012 for securing tip 52. When engaged, lip 1012 fits into groove 1040 of tip 52. Although tip 52 and neck 1010 can be made from almost any material, both are preferably made from plastic or rubber. In one application, the parts are molded using two durometers so that a softer material is used to produce tip 52. For example, an outer surface of tip 52 is generally a soft material that conforms to an inner ear canal while neck 1010 is a harder rubber to which tip 52 can be firmly attached. Additionally, a hard rubber is used on an inner portion of tip 52, while outside portion of tip 52 exposed to ear canal can be a softer rubber. Thus, tip 52 can be firmly attached to neck 1010, which is a hollow tube-like structure to guide sound from speaker 109 into patient's 72 ear canal.

One aspect of the present invention involves providing a cord 1050 for removing or engaging a component of hearing aid test unit 20. For instance, cords 1050 are connected to removably attached hearing aid component such as tip 52. One purpose of the cord 1050 is to provide a method of removing tip 52 from an ear canal if, upon removal of hearing aid test unit 20, tip 52 detaches and becomes lodged in patient's ear canal.

Another purpose of cord 1050 is to provide a mechanism in which a removably attached component can be engaged with another component of hearing aid test unit 20, 22. For instance, an audiologist can pull on cord 1050 to engage tip 52 onto neck 1010. In one application, pull cord 1050 is dental floss or the like. Generally, cord 1050 can be made of any suitable material to which a force can be applied.

In an alternate embodiment as previously discussed, shell 54 is a sheath of plastic that optionally includes a cord so that an audiologist can pull the sheath over core 50 for use by a new patient. A sheath is preferably a thin plastic skin and is similar in respects to those used for thermometers.

It should be noted that nest 70 and neck 1010 are optionally a unique component separately manufactured from shell 54. Neck 1010 and tip 52 can be combined as a single assembly including pull cord 1050. Shell 54 can also include a pull cord 1050.

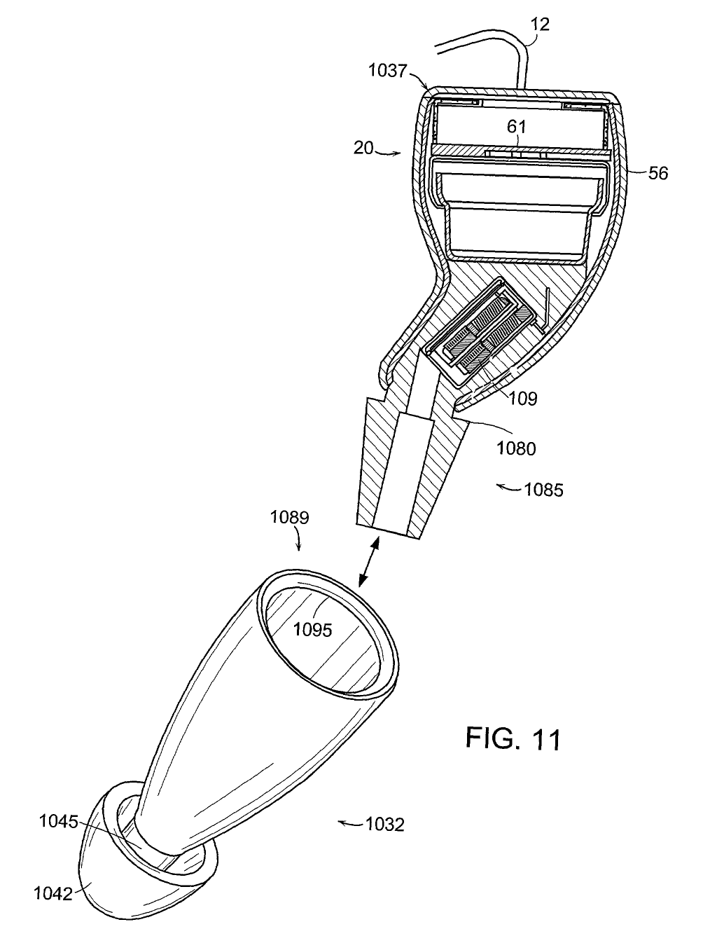

FIG. 11 is a cross-sectional view of a disposable sheath and integrated tip according to certain principles of the present invention.

As shown, hearing aid test unit 20 or any other hearing aid device can include a permanent shell 56 that is then covered by a disposable and removably attached sheath 1032. Opening 1089 of sheath 1032 can be pulled over tip 1085 to cover shell 56 so that successive users tested with hearing aid test unit 20 are not exposed to germs of a previous user. For example, sheath 1032 can be thrown away so that hearing aid looks and feels new again for reuse. Accordingly, a removably attached component such as sheath 1032 disposed in relation to a body of the hearing aid device 20 can be replaced for use by other patients. In a specific application, the removably attached component in relation to the body of the hearing aid device is used to cover a substantial portion of hearing aid test unit 20. Depending on a user, a significant amount of ear wax can be deposited on sheath 1032 during use.

Although not shown, sheath 1032 is optionally used in conjunction with a hearing aid device that does not include cable 12. For example, a sheath 1032 can be fitted over a dispensed hearing programmed to a fixed acoustical format.

To aid the process of attaching sheath 1032, cords such as dental floss are optionally integrated with sheath 1032 or attached to sheath 1032 for pulling sheath completely over shell 56.

When a sheath 1032 and hearing aid device are engaged, a mushroom-shaped tip of sheath 1032 provides a means for snugly fitting a hearing aid into an ear canal of a user. Preferably, mushroom tip 1042 is integrally attached to sheath 1032 that is formed using a combination of material such as soft rubber or plastic composites. Additionally, tip 1042 is preferably a soft rubber so that it conforms the shape of an ear canal.

In an alternate embodiment, sheath 1032 is formed so that it does not include mushroom tip 1042. However, sheath 1032 is designed to cover a substantial portion of hearing aid test unit 20. A removably attached tip 52 as shown in FIG. 10 can be engaged with neck 1085. Thus, hearing aid test unit 20 can include multiple removably attached components.

In the embodiment as shown in FIG. 11, a ridge or groove (not shown) can be formed on the inside of neck 1045 so that the groove can be engaged with angled tip 1085 including lip 1080. Consequently, tip 1042 and generally sheath 1032 is snugly secured to sheath 1032.

Two or more durometers can be used to produce sheath 1032 so that a softer material is used to form tip 1042. Preferably, tip 1042 is made of a soft material so that it easily conforms to the shape of an ear canal. Body of sheath 1032 is optionally made from thin rubber sheet material that fits snugly to the body of hearing aid test unit 20. Consequently, forces of the sheath against the body of hearing aid test 20 unit help to keep sheath 1032 secured to hearing aid test unit 20.

In a specific application, sheath 1032 can be unrolled like a condom over body of sheath 1032 so that it is easier to engage sheath 1032 with hearing aid test unit 20.

Sheath 1032 can also include a ridge or lip 1095 on the inside of opening 1089 so that sheath 1032 fits snugly secured over microphone end 1037 of hearing aid. Thus, sheath 1032 is less likely to disengage and fall off during use or extraction from an ear canal. If sheath 1032 is accidentally lodged in ear canal of a user, it generally can be removed by pulling on body of sheath 1032 itself. That is, a core hearing aid test unit optionally including a shell 56 can be dislodged from an ear canal by pulling on cable 12. A sheath 1032 can become disengaged form shell 56 and remain partly lodged within an ear canal of a user. Generally, sheath 1032 can be removed simply by pulling on a portion of the sheath extending out of an ear canal.

It should be noted that cable 12 can be used as a pull cord instead of disposing an extra assembly onto a body of hearing aid test unit 20. For example, a patient can remove hearing aid test assembly lodged in an ear canal by pulling on cable 12. Thus, cable 12 be provided for multiple purposes. First, a hearing aid device can be reprogrammed via signals received over cable 12. Second, the end of cable 12 can be used to pull hearing aid test unit 20 out of an ear canal after use.

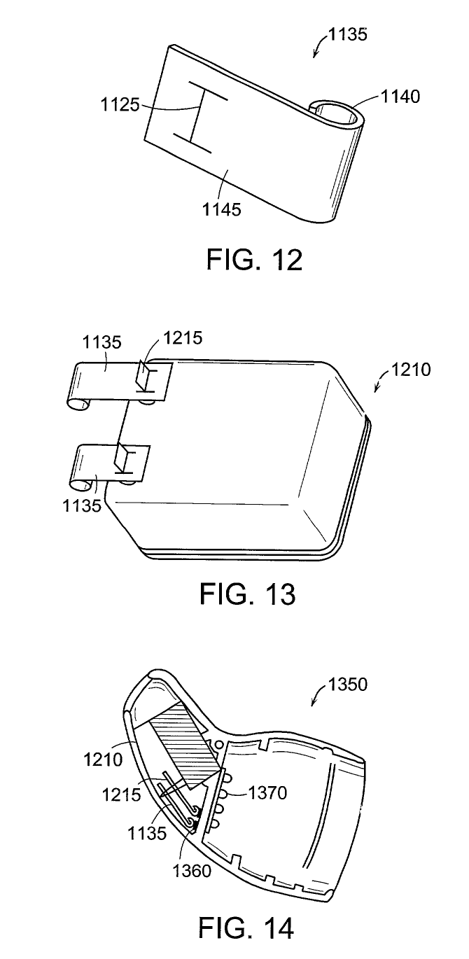

FIG. 12 is a diagram of an electrically conductive strip according to certain principles of the present invention.

As shown, conductive strip 1135 is manufactured from a flat metal strip although any suitable shape generally can be used. Slit 1125 is stamped or etched to produce an opening on strip 1135. A curl 1140 is formed at one end of strip 1135 by appropriately bending the malleable strip 1135. Notably, curl 1140 can be other suitable shapes to make contact with a corresponding electrode.

Copper can be used to produce conductive strip 1135 and a coating such as solder is optionally applied to the surface to reduce the effects of oxidation. Thus, a better connection can be achieved when conductive strip 1135 is attached to provide a connection between two terminals. Once attached to a protruding terminal, glue such as epoxy can be applied to the terminal so that strip 1135 does not fall off.

FIG. 13 is a diagram illustrating an embodiment in which a conductive strip is attached to a protruding terminal according to certain principles of the present invention.

Slit 1125 of strip 1135 preferably includes a tongue. For example, when slit 1125 is shaped like the letter “H” as shown, two tongues are produced. Of course, slit 1135 is optionally a single line or, alternatively, half of a letter “H” to produce one tongue. Generally, slit 1135 can be any suitable shape to accept a protruding terminal 1215.

Component 1210 such as speaker 109 includes one or more protruding terminals 1215 to which a conductive strip 1135 is attached. The method of attaching the lead is somewhat simple. For example, slit 1125 is lined up with terminal 1215 and pressure is then exerted so that terminal 1215 is forced through slit 1125 as shown. Preferably, the width of slit 1125 is smaller than the corresponding width of terminal 1215. Thus, the two opposing tongues exert a force against terminal 1215 so that strip 1135 is removably attached to protruding terminal 1215 of component 1210. Solder or glue is optionally applied to further secure strip 1135 to terminal 1215.

FIG. 14 is a cross-sectional view of a hearing aid device illustrating how a conductive strip can provide connectivity between two nodes according to certain principles of the present invention.

Conductive strip 1135 attached to terminal 1215 of component 1210 provides connectivity to electronic circuitry 1370 disposed within hearing aid device 1350. More particularly, a conductive path is formed by a connection between terminal 1215 and removably attached strip 1135. Curl 1140 at end of strip 1135 is then disposed to touch an electrode 1360 such as an electrically conductive pad of electronic circuitry 1370.

Although shapes other than a curl can be used to provide a contact point for electrode 1360, use of a curl 1140 can be beneficial because it optionally provides a spring like effect so that strip 1135 provides a good contact with electrode 1360 without applying too much pressure. Strip 1135 is preferably bendable, providing an additional spring-like effect. Curl 1140 also enables strip 1135 to contact electrode 1360 at almost any angle at which it is disposed. Consider that curl 1140 is round surface upon which a contact can be made at any point.

Hearing aid device 1350 can include a plastic housing or shell to retain its components and post or guides are optionally included in the plastic housing so that curl 1140 of conductive strip 1135 provide a connection with electrode 1360. More specifically, guides or posts can be strategically placed to hold a strip 1135 in place ensuring that a proper force is exerted strip 1135 to connect electrode 1215 with pads 1360.

This method of providing connectivity reduces assembly time and increases reliability. For instance, it is not necessary to solder wire leads to terminal 1215 for supporting connectivity. Reliability is increased because it is not necessary to heat terminal 1215 for attaching a lead. The process of heating a lead of component 1215 potentially causes stress and damage to the component 1215.

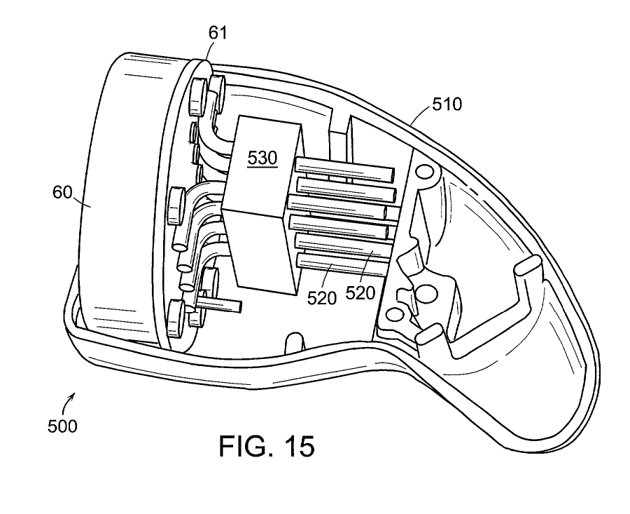

FIG. 15 is a cross-sectional view of a hearing aid device including a connector assembly according to certain principles of the present invention.

As shown, microphone 60 is bonded to electronic circuitry 61 for supporting an acoustical response of hearing aid device 500. An acoustical input is sensed via microphone 60 and a corresponding electrical signal is amplified by electronic circuit 61. In addition to amplification, the acoustical input signal is filtered according to a selected acoustical format to produce an output signal to drive a speaker device.

Surface mount connector device 530 is attached to electronic circuit 61 so that corresponding signals are more easily accessible for coupling to other devices. For example, one output of electronic circuit 61 is used to drive a receiver or speaker device eventually populated in hearing aid 500. A connection is typically made by connecting receiver to appropriate leads 520. Other leads 520 of connector 530 can be used to support testing of electronic circuit 61. This is generally achieved by putting the hearing aid device 500 in different modes and measuring responses at appropriate test points. Additional details regarding testing and format selection are provided in copending U.S. application Ser. No. (2506.2019-001), previously incorporated by reference. Based on the principles as previously discussed, connector 530 supports access to nodes on electronic circuit 61 without having to solder individual leads to corresponding test points.

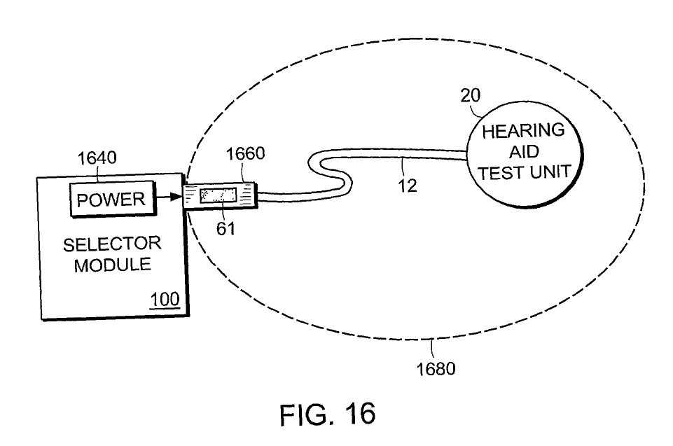

FIG. 16 is a block diagram of a hearing aid test unit including electronic circuitry disposed in a cable assembly according to certain principles of the present invention.

As previously discussed, selector module 100 generates a command to select an acoustical format of hearing aid test unit 20. Electronic circuit 61 then decodes the received command for setting an acoustical response of the hearing aid test unit 20. However, in the embodiment shown, electronic circuitry 61 normally disposed in hearing aid test unit 20 as previously discussed is instead disposed between selector module 100 and hearing aid test unit 20.

Generally, connector module 1660 including electronic circuit 325, cable 12 and hearing aid test unit 20 is assembled as a single unit for plugging into selector module 100. Thus, cable assembly 1680 including multiple electronic components is preferably tested and programmed so that hearing aid test unit 20 supports a corresponding set of acoustical formats as previously discussed in FIG. 6.

One aspect of the present invention involves trimming a hearing aid test unit 20 so that an acoustical output of hearing aid test unit 20 conforms to a standard. Electronic test circuit 61 is trimmed by programming it with compensation factors to account for component variations.

In one application, electronic circuit board 61 includes a flexible circuit board so that it can be disposed in a small space. For example, electronic circuit 61 such as a flexible circuit board can be disposed in connector 1660 or cable 12.

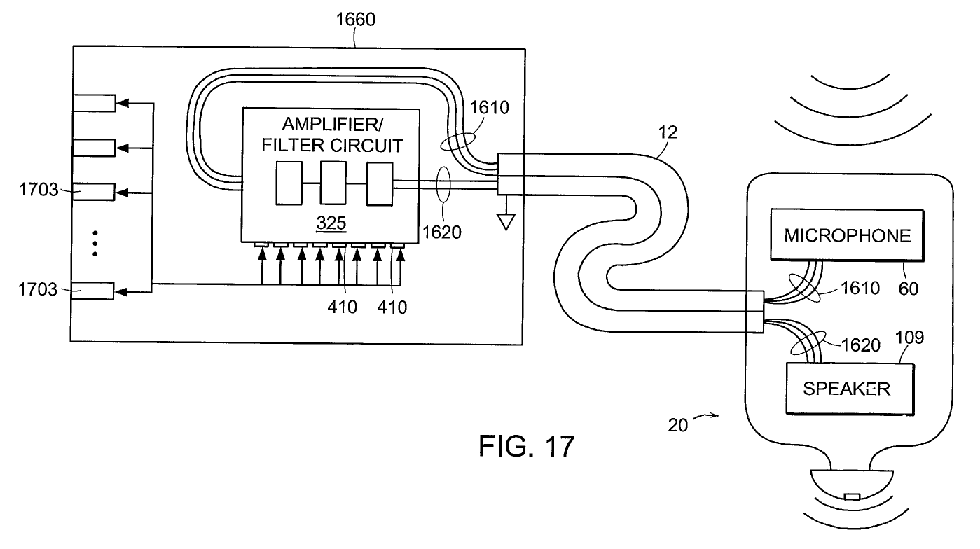

FIG. 17 is a more detailed block diagram of an electronic circuit disposed in a connector module according to certain principles of the present invention.

As shown, microphone 60 disposed in hearing aid test circuit 20 is tethered to connector module 1660 via cable 12 and, more specifically, wires 1610. Wires 1610 provide a conductive path for carrying a detected acoustical input signal from microphone 60 to electronic circuit 325. Shielding is optionally provided on wires 1610 for signal integrity.

As previously discussed, electronic circuit 61 amplifies and filters the input signal from microphone 60 to produce an output signal that is transmitted to speaker 109 via wires 1620 of cable 12. Speaker 109 generates a sound output based on a detected acoustical input at microphone 60.

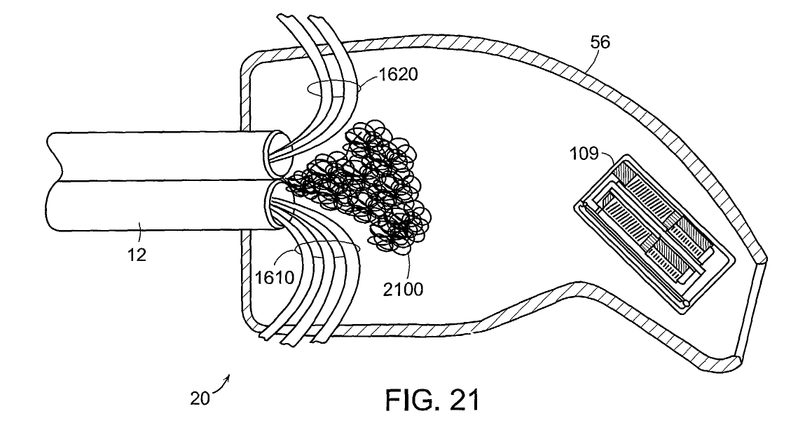

Since electronic circuit 61 is not disposed in hearing aid test unit 20, this space can otherwise be used to properly terminate cable 12. For example, a shield of cable 12 can be terminated at hearing aid test unit 20 by gluing the shield wires to a shell of hearing aid test unit 20. See FIG. 21 and related text for more details.

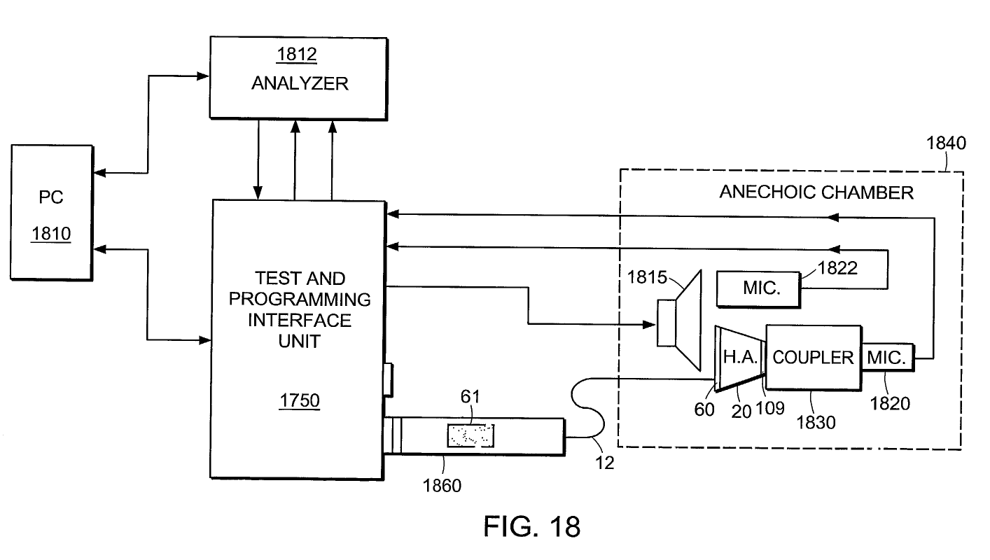

FIG. 18 is a block diagram of a test unit for trimming a cable assembly according to certain principles of the present invention.

As previously discussed, electronic circuit 61 is trimmed to account for the characteristics of microphone 60, electronic circuit 61, and speaker 109 so that its output conforms to a standard. Consequently, a particular acoustical format generally will be identical for each of multiple hearing aid devices, whether such devices are fixed or re-programmable.

Electronic circuit 61 is tested by generating a sound output from speaker 1815 at a specified volume into microphone 60 of hearing aid test unit 20. Based on a detected acoustical input at microphone 60, an output is generated at speaker 109 of hearing aid test unit 20.

The acoustical output of speaker 109 is then measured to determine an acoustical response of hearing aid test unit 20. Based on a measured acoustical response, a compensation factor is stored in memory 470 of acoustical circuit 61 so that the acoustical formats provided by hearing aid test unit 20 conform to a standard. Consequently, an acoustical response of hearing aid test unit 20 for selecting a desired acoustical format provides the same sound quality as a production hearing aid dispensed to a patient even though electronics 61 is disposed in connector 1860 or cable 12.

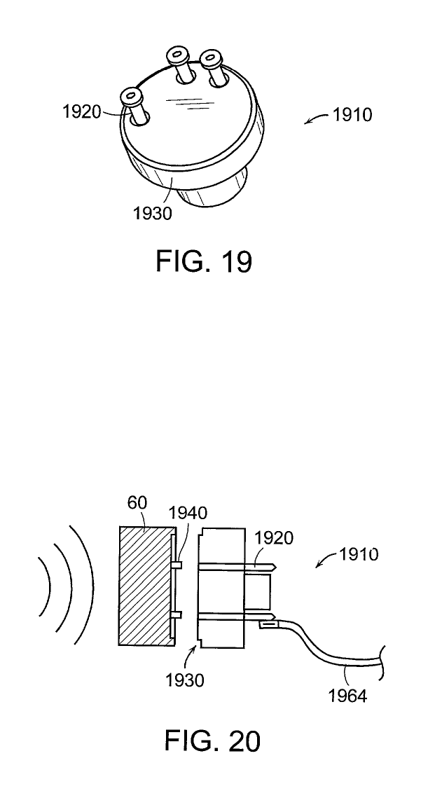

FIG. 19 is a diagram of a microphone socket assembly according to certain principles of the present invention. As shown, socket 1910 includes a cylindrically shaped socket including a terraced step 1930.

FIG. 20 is a detailed cross-sectional diagram of a microphone and corresponding socket according to certain principles of the present invention.

Terminals 1940 of microphone 60 can be difficult to solder because of their small size. Consider that a hearing aid test unit 20 can be small enough to fit in an ear.

To simplify a process of connecting microphone 60 to other circuitry, microphone 60 can be engaged with socket 1910 to form an assembly. Thus, terminals 1940 of microphone 60 can be electrically connected to lead 1964 and, optionally, cable 12 for transmitting a signal to a target device such as electronic circuit 325.

One aspect of socket 1910 is its non-conductive body that engages with a portion of an electronic component such as microphone 60. Microphone 60 is optionally conductive or non-conductive depending on a particular application.

As shown, a body socket 1910 itself can be a socket for mating with another component. For example, socket 1910 optionally includes a terraced step 1930 that engages with cavity of microphone 60. Other suitable male-female mating configurations can be employed to engage socket 1910 and microphone 60. As a result of forming this structure, microphone 60 is less likely to be damaged and it is easier for an assembler to attach leads directly to pins 1920 rather than terminals 1940 of microphone 60.

When engaged, terminals 1940 of microphone 60 are less likely to be damaged during the assembly process since the terminals are no longer exposed and the connection resulting from the engagement of socket 1910 and component form a natural shield so that potentially fragile terminals 1940 will not be damaged as a result of mishandling. More specifically, externally exposed bodies of socket 1910 and microphone 60 will bear the brunt of potentially destructive forces caused by mishandling.

Microphone socket 1910 can include multiple pin receptacles 1920 for receiving terminals 1940 of microphone 60. Each pin receptacle 1920 is generally a conductive rod including a receptacle that is pushed through a corresponding hole bored through body of socket 1910.

An end of pin receptacle 1920 disposed towards microphone 60 includes a socket for receiving a corresponding terminal of microphone 60. At an opposite end, pin receptacle 1920 is generally a metallic post or rod that extends through body of socket 1910 for soldering lead 1964.

Based on the socket/microphone assembly as discussed, socket and component are potentially engaged in two ways. First, a non-conductive body of the socket and component can be engaged. More specifically, socket 1910 can include a terraced step 1930 so that microphone 60 can be removably engaged with socket 1910 to form a single assembly. Second, conductive parts such as terminals 1940 of microphone 60 can be engaged with pin receptacles 1920 of socket 1910 to provide electrical connectivity between the assembly and other circuit to which the socket is connected.

To ensure that the microphone and socket 1910 are not accidentally misaligned during assembly, conductive terminals 1940 and corresponding pins 1920 of socket 1910 can be asymmetrically disposed to form a pattern so that each conductive terminal plugs into corresponding receptacle when the socket 1910 and component such as microphone 60 are properly aligned. Consequently, terminal 1940 of a microphone 60 that would otherwise be damaged during assembly is protected when microphone 60 is properly engaged with socket 1910.

FIG. 21 is a cross-sectional diagram of a cable termination according to certain principles of the present invention.

Cable 12 is terminated in hearing aid test unit 20 to provide strain relief. As shown, cable 12 includes wires 1610, 1620, microphone 60 and speaker 109 respectively. One method of providing strain relief is to attach shield wires 2100 to form termination 2100 to shell 56 of hearing aid test unit 20.

In one application, housing of hearing aid test unit 20 is a solid metallic shell 56 or conductively coated shell 56, rather than a plastic shell 56, and shield wires 2100 are attached to shell 56 via conductive epoxy or solder. In this way, hearing aid test unit 20 can be isolated so that it neither conducts nor radiates electromagnetic radiation. Consequently, electronics disposed in the hearing aid test unit 20 will not interfere with other electronic devices nor will hearing aid test unit 20 be as susceptible to external radiation sources that might other wise interfere with its operation.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of selecting an acoustical format for a hearing impaired user, the method comprising the steps of:
providing a hearing aid test unit that simulates a production hearing aid to be supplied to a user; and
coupling a selector module to the test unit whereby the user may select one of multiple electroacoustic formats.

2. A method as in claim 1, wherein the hearing aid test unit includes a disposable shell and tip.

3. A method as in claim 1, wherein the hearing aid test unit includes a disposable tip that is used to direct amplified sound into an ear canal of the user.

4. A method as in claim 1, wherein a core of the hearing aid test unit includes a microphone and speaker, and the core is removably attached to a shell.

5. A method as in claim 1, wherein the hearing aid test unit includes a reusable core and a disposable shell and tip.

6. A method as in claim 1, wherein the core and the shell are removably attached by a tab inserted through a lock mechanism of the shell, a portion of the lock mechanism being broken when the shell and tip are separated from the core.

7. A method as in claim 1, wherein the hearing aid units are provided for both a right and left ear of a user to test different electroacoustic formats.

8. A method as in claim 1, wherein a selectable electroacoustical format is provided in a mass produced hearing aid device having a form, fit and function similar to the hearing aid test unit.

9. A method as in claim 1 further comprising the step of:
pressing a keypad to select one of multiple electro acoustical formats.

10. A method as in claim 9 further comprising the step of:
providing multiple types of sample recordings so that a user can compare one format to another.

11. A method as in claim 1 further comprising the step of:
downloading digital information from the selector module to program a corresponding acoustical format of the hearing a id test unit.

12. A selector module comprising:
a housing in which an electronic circuit is enclosed; and
a controller for selecting one of a plurality of signal processing formats such that a user may select an optimum format for use in a corresponding production hearing aid device.

13. A selector module as in claim 12, wherein the formats are downloaded from the selector module into the hearing aid device.

14. A selector module as in claim 12, wherein the formats are encoded in the hearing aid device and the selector module provides a signal for selecting a particular format.

15. A selector module as in claim 12, wherein a set of coefficient multipliers are used to define a frequency response of the hearing aid device for a particular format.

16. A selector module as in claim 12, wherein the selector module is portable.

17. A selector module as in claim 12 further comprising:
a keypad input for selecting a particular acoustical format.

18. A hearing aid test unit for simulating a form and fit of a corresponding production hearing aid device, the hearing aid test unit comprising:
a core including a microphone to sense a sound input and an electronic circuit to drive a speaker based on the sound input; and
a cable including an electronic signal for selecting a frequency response of the hearing aid test unit device.

19. A hearing aid test unit as in claim 18, wherein the hearing aid test unit electronic circuit is powered through the cable.

20. A hearing aid test unit as in claim 18 further comprising:
a shell for housing the core; and
a detachable tip for directing sound generated by the speaker into an ear canal.

21. A hearing aid test unit as in claim 20, wherein the tip is separable from the core such that the core may be re-used with another tip.

22. A hearing aid test unit as in claim 20, wherein the shell is adapted to be inserted in an ear of a typical user.

23. A hearing aid test unit as in claim 20, wherein the shell is separable from the core by breaking a portion of the shell, after which the shell cannot be re-attached to the core.

24. A hearing aid test unit as in claim 18, wherein a frequency response is selected by pressing a keypad corresponding to an acoustical format.

25. A hearing aid test unit as in claim 24, wherein a frequency response is selected from a matrix of acoustical formats.

26. A hearing aid test unit as in claim 22, wherein the matrix includes N×M acoustical formats, where N and M are both at least equal to 2.

27. A hearing aid test unit as in claim 24, wherein a cover is provided over the keyboard to prevent access to some keypads while the cover is closed.

28. A hearing aid test unit as in claim 18, wherein the cable includes a telephone connector.

29. A hearing aid test unit as in claim 28, wherein a user compares multiple formats to select an optimal format for use in a corresponding production hearing aid device.

30. A hearing aid device comprising:
a core including a microphone and speaker to sense and amplify a sound input for a hearing impaired patient; and
a removably attached component disposed in relation to a body of the core of the hearing aid device.

31. A hearing aid device as in claim 30, wherein the removably attached component is a sheath protecting the core of the hearing aid device from exposure to human tissue.

32. A hearing aid device as in claim 30, wherein the removably attached component includes a pull cord.

33. A hearing aid device as in claim 30, wherein the removably attached component is a disposable tip for directing sound into an ear of the hearing impaired patient.

34. A hearing aid device as in claim 32, wherein the pull cord of the removably attached component is provided so that a patient can dislodge said component from an ear.

35. A hearing aid device as in claim 30, wherein the pull cord of the removably attached component is provided so that the component can be secured to the core of the hearing aid device.

36. A hearing aid device as in claim 30, wherein the removably attached component is a shell for housing the core of the hearing aid device and the shell includes a locking mechanism for securing the component to the core.

37. A hearing aid device as in claim 36, wherein an indicator is provided to identify that the shell was previously used.

38. An electrically conductive lead comprising:
a conductive strip; and
a slit disposed on the conductive strip for attaching said conductive lead to an electrode.

39. An electrically conductive lead as in claim 38, wherein the slit is shaped to form at least one tongue.

40. An electrically conductive lead as in claim 38, wherein at least part of the strip is flat.

41. An electrically conductive lead as in claim 38, wherein the strip is used to electrically connect a terminal of a leaded electronic component to electronic circuitry disposed in a hearing aid device.

42. An electrically conductive lead as in claim 38, wherein the strip includes three slits that are disposed to form a letter H.

43. An electrically conductive lead as in claim 38, wherein the strip is attached to a protruding terminal by inserting the protruding terminal through the slit in the conductive strip.

44. An electrically conductive lead as in claim 38, wherein the slit is formed by an etching process.

45. An electrically conductive lead as in claim 38, wherein the slit is formed by a stamping process.

46. An electrically conductive lead as in claim 38, wherein the curled end of the conductive strip is disposed to provide force on an electrode.

47. An electrically conductive lead as in claim 38, wherein one end of the conductive strip is curled.

48. An assembly comprising:
an electronic circuit for processing an acoustical input;
a connector attached to the circuit; and
a shell housing the connector and the electronic circuit to form a hearing aid device that fits in an ear.

49. An assembly as in claim 48, wherein a cable is attached to the connector.

50. An assembly as in claim 48, wherein the connector is a surface mount connector.

51. An assembly as in claim 48, wherein the connector includes conductive posts to which at least one wire is soldered for connecting a lead.

52. An assembly as in claim 48, wherein the connector includes at least one conductive pin receptacle.

53. An assembly as in claim 48, wherein the electronic circuit includes an amplifier to amplify an acoustical input of the hearing aid.

54. An assembly as in claim 48 further comprising:
a transducer to which the electronic circuit is attached.

55. An assembly as in claim 54, wherein the transducer is a microphone for detecting an acoustical input.

56. An assembly as in claim 54, wherein the hearing aid device includes a speaker for generating an acoustical output.

57. An assembly as in claim 48, wherein the connector is a socket.

58. An apparatus comprising:
a cable assembly terminated at one end by a hearing aid device; and
an electronic circuit disposed in the cable assembly for processing an acoustical input of the hearing aid device to produce an acoustical output.

59. An apparatus as in claim 58, wherein a housing for the electronic circuit is disposed at a point along a length of the cable assembly.

60. An apparatus as in claim 58 further comprising:
a selector module for selecting a mode of the electronic circuit and acoustical response of the hearing aid device.

61. An apparatus as in claim 60, wherein the cable is connected to the selector module by a connector at an end of the cable.

62. An apparatus as in claim 60, wherein the selector module includes a keypad for selecting an acoustical response of the hearing aid device.

63. An apparatus as in claim 58 further comprising:
a microphone disposed in the hearing aid device for detecting the acoustical input, whereby a signal corresponding to the acoustical input is transmitted over at least a portion of the cable to the electronic circuit for processing.

64. An apparatus as in claim 63 further comprising:
a speaker disposed in the hearing aid device that is driven by the electronic circuit to produce an amplified output signal.

65. An apparatus as in claim 58, wherein the electronic circuit is disposed in a connector at an end of the cable assembly opposite the hearing aid device.

66. An apparatus as in claim 58, wherein the electronic circuit includes a flexible circuit board disposed in the cable assembly.

67. An apparatus as in claim 58, wherein the electronic circuit can be programmed to one of multiple acoustical formats, each of which defines a response of the hearing aid device for an entire range of audible inputs detectable by a human ear.

68. An apparatus as in claim 58, wherein the electronic circuit includes an amplifier and a filter circuit for processing an acoustical input of the hearing aid device to generate an acoustical output of the hearing aid device.

69. An apparatus as in claim 58, wherein shield wires disposed in the cable assembly are terminated at one end by being glued to a component of the hearing aid device.

70. An apparatus as in claim 58, wherein the electronic circuit is tested and trimmed so that an acoustical output of the hearing aid device conforms to a standard for a range of acoustical inputs.

71. An apparatus as in claim 70, wherein trim information is stored in memory of the electronic circuit to compensate for a variation of a component disposed in the hearing aid device.

72. An apparatus as in claim 58, wherein the hearing aid device is used to test a hearing impaired patient that selects a preferred acoustical format, and a corresponding untethered hearing aid device programmed to a fixed format is dispensed to the patient.

73. An apparatus as in claim 72, wherein the untethered hearing aid device is disposable.

74. An assembly for use in a hearing aid device that fits in an ear, the assembly comprising an electronic component and a socket, whereby a portion of the electronic component is formed to engage with a non-conductive portion of the socket.

75. An assembly as in claim 74, wherein the electronic component is a transducer.

76. An assembly as in claim 74, wherein the electronic component is a microphone.

77. An assembly as in claim 74, wherein the electronic component includes protruding conductive terminals and the socket includes receptacles for receiving the protruding conductive terminals.

78. An assembly as in claim 77, wherein the conductive terminals are asymmetrically disposed on the socket so that each conductive terminal is plugged into a corresponding receptacle when the socket is engaged with the electronic component.

79. An assembly as in claim 74, wherein the socket is cylindrically shaped and includes a terraced step for engaging with the electronic component.

80. An assembly as in claim 74, wherein the receptacles are pins that extend through a body of the socket so that a lead can be attached to a particular pin.
